(19) United States
(12) Patent Application Publication
(10) Pub. No.: US 2014/0024884 A1
(43) Pub. Date: Jan. 23, 2014

(54) GASTRIC BAND CONTROLLED BY AUTOMATIC EATING DETECTION

(71) Applicant: Edy E. Soffer, Los Angeles, CA (US)

(72) Inventor: Edy E. Soffer, Los Angeles, CA (US)

(21) Appl. No.: 13/944,574

(22) Filed: Jul. 17, 2013

Related U.S. Application Data
(60) Provisional application No. 61/672,674, filed on Jul. 17, 2012.

(51) Int. Cl. A61F 5/00

(52) U.S. Cl. CPC A61F 5/005
USPC 600/37

(57) ABSTRACT

A system, apparatus, and method for controlling a gastric band in response to the detection of eating in a subject. The system includes an inflatable/deflatable gastric hand, a monitoring unit that monitors electrical activity changes of the subject's lower esophageal sphincter, and a hydraulic system that moves fluid into and out of the gastric band in response to a signal from the monitoring unit about the electrical activity changes. The method includes moving fluid into and out of an inflatable/deflatable gastric band in response to changes in electrical activity of the subject's lower esophageal sphincter.

### BACKGROUND

1. Field of Invention

This invention relates to systems and methods, including those utilizing automatic eating detection to control a gastric band for various purposes, including the treatment of obesity.

2. Related Art

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Currently, gastric bands in use for bariatric surgery consist of silastic tubes wrapped around the stomach. They can be adjusted to be tightened or loosened, by increasing or decreasing the volume of fluid within the band. All bands in current use need to be kept at a certain tightness, so as to interfere with food intake in order to achieve weight loss. However, complications, such as acid reflux and esophageal dilatation, related to band tightness can occur and require either loosening of the band with subsequent weight gain, or removal of the band. Band replacement may also be required.

### SUMMARY

In one aspect, a system for controlling a gastric band in a subject is provided. The system includes an inflatable/deflatable gastric band, a monitoring unit that monitors electrical activity changes of the subject's lower esophageal sphincter, and a hydraulic system that moves fluid into and out of the gastric band in response to a signal from the monitoring unit about the electrical activity changes. The hydraulic system can comprise a pump for moving the fluid, and one or more reservoirs for holding the fluid. In some embodiments, the hydraulic system can comprise a high pressure reservoir fluidly connected to the gastric band, a low pressure reservoir fluidly connected to the gastric band, and a pump for moving fluid between the high and low pressure reservoirs.

The monitoring unit, the hydraulic system, or both, can be implantable on or within the subject.

The monitoring unit can comprise a microprocessor that monitors electrical activity and detects eating and/or generates electrical signals based on the monitored electrical activity.

In some embodiments, the system can include a computer for receiving electrical signals, analyzing electrical signals, processing electrical signals, and sending a signal regarding the electrical signals to another system, computer or device. In some embodiments, the system can include one or more electrodes for detecting the electrical activity changes and positionable within, in contact with or proximate to the gastroesophageal junction of the subject.

Electrical activity changes can indicate that the subject has started consuming food or drink, is consuming food or drink, has stopped consuming food or drink, has consumed food or drink, or any combination thereof. In some embodiments, an increase in amplitude of the electrical activity changes to a value greater than baseline indicates food or drink intake. In some embodiments, an about three to about four fold increase in amplitude of the electrical activity changes from baseline indicates food or drink intake.

In another aspect, a method of controlling a gastric band in a subject is provided. The method includes moving fluid into and out of an inflatable/deflatable gastric band in response to changes in electrical activity of the subject's lower esophageal sphincter. The method can include monitoring the changes in electrical activity prior to moving the fluid. In some embodiments, the monitoring can include monitoring the electrical activity by use of a microprocessor, or monitoring the electrical activity by use of one or more electrodes positioned within, in contact with or proximate to the gastroesophageal junction of the subject, or a combination thereof.

In the method, changes in the electrical activity can indicate that the subject has started consuming food or drink, is consuming food or drink, has stopped consuming food or drink, has consumed food or drink, or any combination thereof. In some embodiments, an increase in amplitude of the electrical activity changes to a value greater than baseline indicates food or drink intake. In some embodiments, an about three to about four fold increase in amplitude of the electrical activity changes from baseline indicates food or drink intake.

The method can be applied to a subject undergoing treatment for obesity or treatment to prevent obesity. The subject can be a human or an animal.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts a schematic representation of various components of the system of adjusting a gastric band in conjunction with eating detection.

FIG. 2 depicts a schematic representation of the computerized hydraulic system and gastric band of the system for adjusting a gastric band in conjunction with eating detection.

FIG. 3 depicts a representative location for detection of the LES electrical activity for use in accordance with a system for adjusting a gastric band in conjunction with eating detection in accordance with various embodiments.

FIG. 4 depicts an example of a prior art gastric band.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application. Priority provisional application 61/672,674, filed on Jul. 17, 2012, is incorporated by reference herein.

One skilled in the art will recognize many methods and materials similar or equivalent to those described herein, which could be used in the practice of the present invention.

There exists a need in the art for systems and methods of adjusting gastric bands to avoid the complications discussed above. Thus, in one aspect, apparatuses, systems and methods for actively adjusting a gastric band in conjunction with food intake are provided. Examples of subjects that can benefit from these apparatuses, systems and methods include but are not limited to subjects who do not have a gastric band, and subjects who have an implanted adjustable gastric band. The former group can have the full treatment, and the latter group can have their passive gastric band upgraded to an active band by addition of the eating detection mechanism and a computerized hydraulic system. A gastric band can be inflated and consequently be tightened at the onset of a meal, but remain deflated most of the time. This can avoid complications and can function in a physiologic manner.

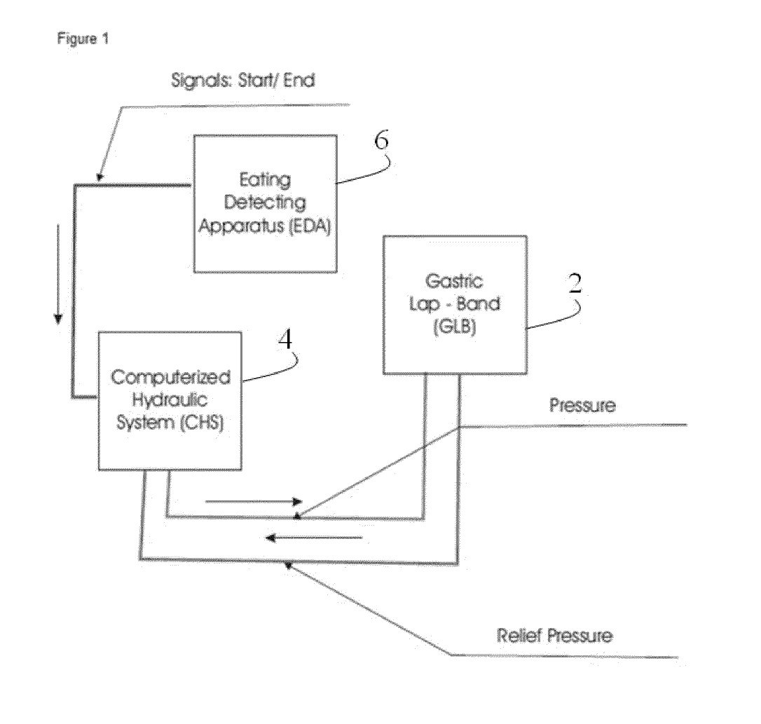

Referring to FIG. 1, embodiments provide for a system, comprising a gastric band 2, a computerized hydraulic sub-system 4 to inflate and deflate the gastric band, and an eating detection sub-system 6.

inflation and deflation tubes are connected to the computerized hydraulic sub-system and to the gastric band to control the obstruction of the stomach in accordance to signals provided by the eating detection sub-system.

The full system can be placed by laparoscopy.

Gastric Band

In various embodiments, the gastric band comprises a lumen. The gastric band is capable of being inflated and deflated. The gastric band can be made of any suitable natural or synthetic material. In various embodiments, the band is an elastomeric band. In various embodiments, the material for the gastric band is an inert polymer. In various embodiments, the material for the gastric band is a non-toxic polymer. In various embodiments, the material for the gastric band comprises polydimethylsiloxane. In various embodiments, the elastomeric gastric band is a flexible silicone elastomeric gastric band (e.g., Silastic™ silicone elastomer).

In various embodiments, the gastric band can be similar to gastric bands currently in use. In other embodiments, the gastric band can be those gastric bands currently in use.

Computerized Hydraulic System

In various embodiments, the computerized hydraulic system can use a microprocessor to control the system. In various embodiments, the computerized hydraulic system will inflate or deflate the gastric band in accordance to a subject's eating process detected by the eating detection sub-system.

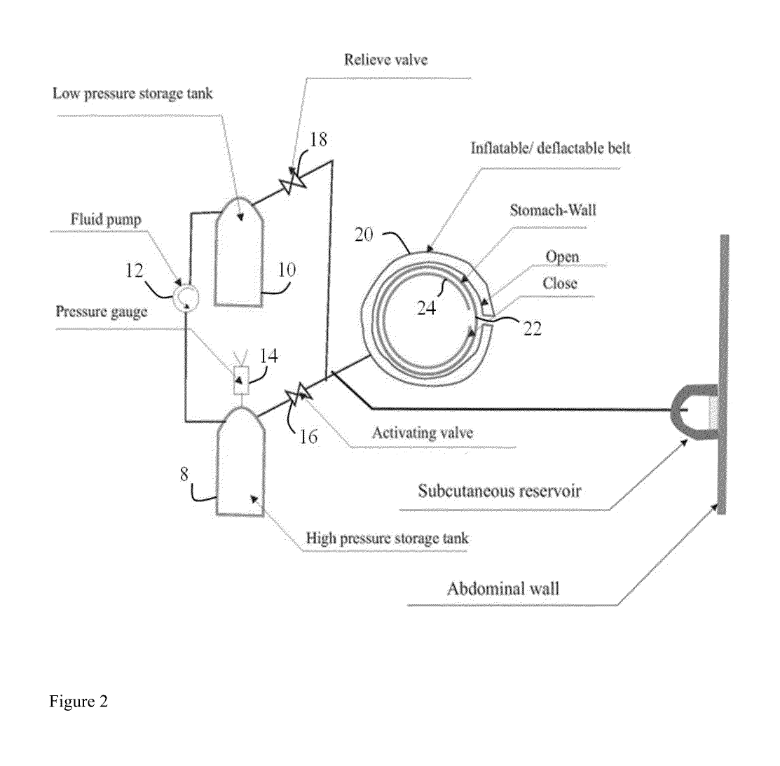

Referring to an embodiment in FIG. 2, the computerized hydraulic system can comprise a first storage tank 8, a second storage tank 10, a fluid pump 12, a pressure gauge 14, a first valve 16 and a second valve 18. In various embodiments, the first storage tank is a high pressure storage tank and the second storage tank is a low pressure storage tank. In various embodiments, the first storage tank and the second storage tank is one unit with a partition between the two storage areas. The high pressure storage tank can be connected to a pressure gauge or a pressure transducer. In various embodiments, the first valve is an activating valve and the second valve is a relief valve. The fluid can be water or another liquid such as a saline solution or another biocompatible liquid.

The microprocessor can detect electrical recording, process algorithms and signals to activate the valves, storage tank(s) and pump(s).

In a further embodiment, the computerized hydraulic system comprises a subcutaneous reservoir. The subcutaneous reservoir can serve as a safety measure. For example, if it is determined necessary, the fluid from the gastric band can be drawn off into the subcutaneous reservoir.

The computerized hydraulic system can have an intervention stage, a stop and recovery stage, and a stand-by stage.

A built-in algorithm controls the computerized hydraulic system to function at the desired stage. The algorithm can be individualized, so as to provide the appropriate detection for each subject. (See Sanmiguel et al. The Effect of Eating on Lower Esophageal Sphincter Electrical Activity, Am J. Physiology 2009; 296: G793-G797.) The first step is eating detection. A change in the amplitude of the electrical activity in the LES region is detected by the implanted electrodes connected to a microprocessor with a dedicated algorithm. The algorithm is based on the fact that the amplitude of electrical activity increases when food in consumed, and is highest with solid food compared to liquids, or swallows of saliva. This detection mechanism can be individualized, and adjusted to the response of each subject. Referring to the embodiment in FIG. 2, when eating is detected, a signal is transmitted to the hydraulic sub-system comprising the first and second storage tanks, pump and valves. After detecting food intake, a signal can be sent by the microprocessor to the activating valve 16 in order to open and then immediately close it, this will result in fluid flowing from the high pressure reservoir 8 to the gastric band 20 to inflate and partially block the stomach. In various embodiments, the signal can be sent by the microprocessor immediately after detecting food intake. In various embodiments, after a preset period of time, which can also be individualized, a signal is sent to the relief valve 18 to open and then immediately close it. This will deflate the band, by allowing fluid to flow from the band to the low pressure reservoir 10. Subsequent to that, the hydraulic pump 12 is switched on to pump the fluid from the low pressure storage tank 10 into the high pressure storage tank 8 until the requested pressure is detected by the pressure gauge 14 which sends a signal, through the microprocessor, to stop the pump. The system is now in the stand-by stage ready to activate again according to a subsequent eating detection signal. In various embodiments, the system can include a miniature inclinometer that helps avoid inflation of the band when the patient is in a supine position, which can indicate sleep. In FIG. 2, stomach wall 22 represents a stomach when the gastric band is in a deflated state; curve 24 represents the partially blocked stomach when the gastric band is in an inflated state.

The intervention stage can comprise a start phase and hold phase. The start phase of the intervention comprises delivering the fluid from the high pressure storage tank to the gastric band to tighten the gastric band and the hold phase of the intervention stage comprises keeping the band tightened. The start phase of the intervention can be quick with a holding period that is variable according to the time need to keep the gastric band in a tightened state. For example, the stall phase of the intervention can be about 1, 2, 3, 4, or 5, seconds. In alternative embodiments, start phase of the intervention can be about 5, 10, 15, 20, 25, 30, 35, 40, 45, 50, 55, or 60 seconds. In various embodiments, the holding period can be the length of a meal. In various embodiments, the holding period can be, but is not limited to, 1, 2, 3, 4, 5, 6, 7, 8, 9, 10, 11, 12, 13, 14, 15, 16, 17, 18, 19, or 20 minutes depending on the duration of the meal. Thus, the holding period can be, but is not limited to 1-5 minutes, 5-10 minutes, 10-15 minutes, 15-20 minutes, 20-25 minutes, 25-30 minutes, 30-40 minutes, 40-50 minutes, 50-60 minutes or longer.

The stop/recovery stage comprises a stop phase and a recovery phase. The stop phase comprises delivering the fluid from the gastric band to the low pressure storage tank to loosen the gastric band. The stopping phase can be quick. For example, the stopping phase can be about 1, 2, 3, 4, or 5, seconds. In alternative embodiments, stopping phase can be about 5, 10, 15, 20, 25, 30, 35, 40, 45, 50, 55, or 60 seconds. The recovery phase comprises delivering the fluid from the low pressure storage tank to the high pressure storage tank. Fluid from the low pressure storage tank is pumped into the high pressure storage tank. The recovery phase can be controlled by the pump capacity to transfer the fluid from the low pressure storage tank into the high pressure storage tank to build the fluid pressure monitored with the pressure gauge in accordance with the need of the stand-by stage. The time of this procedure can depend on various parameters, including but not limited to pump capacity and electrical power.

The stand-by stage allows for the gastric band to remain at a loosened state until the intervention stage is started again.

Embodiments also provide for a method of controlling a gastric band. The method can comprise providing a system comprising a gastric band, a computerized hydraulic sub-system to inflate and deflate the gastric band, and an eating detection sub-system; and placing the system in a subject. The method can further comprise: detecting food ingestion using the eating detection sub-system; and having the computerized hydraulic system initiate the intervention stage. The method can further comprise: detection of the cessation of food ingestion. The method can further comprise: having the computerized hydraulic system be in the stand-by stage.

Automatic Detection of Eating

The eating detection sub-system can act as a controller of the system. After the detection of eating, an algorithm starts a time controlled program, with a variable time delay and sends a signal to the computerized hydraulic system to inflate the gastric band. The band can remain inflated for variable periods of time, which can be adjusted to the eating habits of individual patients. At the end of this preset period of time the band will automatically be deflated. For example, the system can be set to keep the band inflated for 30 min after the detection of eating, and then to deflate. A subsequent eating detection will again inflate the pump for another period of 30 min. In a different embodiment the period of inflation could be 40, 50 or 60 minutes, or other durations as required per individual eating habits.

The system can also be set to keep the band inflated for various periods of time, per individual specifications.

Various embodiments utilize systems and methods of automatic detection of eating to initiate the control of the gastric band. In various embodiments, the automatic detection of eating can utilize the methods, devices, and systems set for the in U.S. Patent Application Publication no. 2010/0076345, which is hereby incorporated by reference as though fully set forth in its entirety.

Electrical activity of the lower esophageal sphincter has been recorded and studied. Swallowing produces changes in the motor activity of the LES. The inventors believed that these changes are related to specific changes in LES electrical activity. The beginning and duration of a meal can be identified by distinct, easily recognizable changes in the amplitude of LES electrical activity. These changes also depend on the type of substance being swallowed (e.g., saliva, liquid and solids), and are most prominent with solid food. Further, during fasting, transient increases in LES electrical activity not related to swallowing do not produce the same increase in electrical activity as seen during swallowing of food. Thus, changes in LES electrical activity can be used for eating detection.

The method of detecting food or drink intake in a subject can comprise: a) placing one or more electrodes in contact with or proximate to the subject's LES; and b) identifying food or drink intake by monitoring electrical activity in or proximate to the LES using one or more electrodes. In various embodiments, bipolar electrodes can be used and thus, only one lead is necessary.

The lower esophageal sphincter is a ring of muscle tissue located at the bottom of the esophagus where the esophagus meets the stomach. Normally, the LES acts as a valve to prevent the backflow of stomach contents into the esophagus. The junction between the esophagus and the stomach is called the gastroesophageal junction.

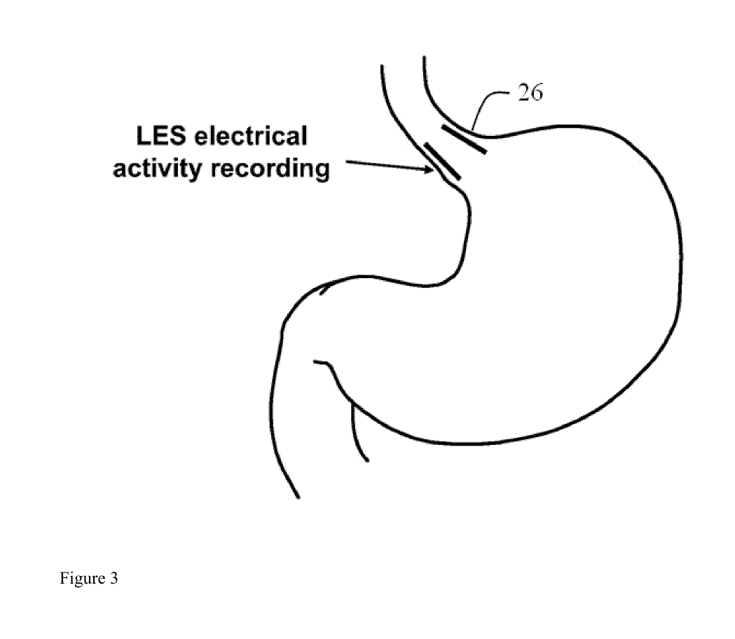

Referring to FIG. 3, in one embodiment, one or more electrodes are placed in contact with the LES 26 or in contact with a proximate region to the LES and the electrical activity is monitored at that location. An increase in the amplitude of electrical activity in the monitored location indicates food or drink intake, and a decrease in the amplitude back to about baseline level indicates the cessation of food or drink intake. Further, the degree of the change in amplitude (e.g., increase in amplitude) can be used to differentiate between types of swallows (saliva, liquid or solid food). The duration of change in amplitude helps to determine the length of period of food consumption. For example, a short duration indicates simple swallows or a very small snack and a long duration indicates the consumption of a larger meal. The electrical activity of the LES while in a resting or non-swallowing state can establish the baseline level, and amplitudes above the baseline can indicate dry swallows, wet swallows, or solid food swallows, depending on the size and duration of the amplitudes.

In some embodiments, a pair of electrodes is placed. Two electrodes can be positioned at opposite sides of the gastroesophageal junction (GEJ). In particular embodiments, one electrode is positioned in the left aspect of the GEJ and a second electrode is positioned in the right aspect of the GEJ.

In other embodiments, one or more electrodes are positioned away from the vagus nerve trunks. In a particular embodiment, one or more electrodes are positioned as far away from the vagus nerve trunks as possible so long as electrical activity indicative of food or drink intake can be detected. In a particular embodiment, two electrodes are positioned as far away from the vagus nerve trunks as possible.

An electrode can be of any size suitable for placement on or in the LES, or on or in a proximate region to the LES. In various embodiments, the electrodes can be about 1 mm long to about 50 mm long, about 5 mm long to about 25 mm long, or about 10 mm long to about 20 mm long. In one embodiment, the electrode can be about 15 mm long. The electrode can also be of any shape suitable for placement on the LES or on a proximate region to the LES; for example, circular, square, rectangular, etc. The electrode can also be of any dimension suitable for placement on the LES or on a proximate region to the LES. The electrode can be attached on the surface of the LES or proximate region, or implanted into the LES or proximate region.

Placing an electrode in contact with the LES or proximate to the LES can be performed by any method known in the art; for example, by a surgical procedure or by an endoscopic procedure. The electrode can be placed on any level in the LES tissue from the inner lining (i.e., mucosa) to the muscle layer. In one particular embodiment, an electrode can be sutured to a muscle layer of the LES or a proximate region to the LES.

In some embodiments, monitoring the electrical activity comprises detecting the electrical activity in the LES. In particular embodiments, monitoring the electrical activity comprise measuring the amplitude and/or duration of the electrical activity in the LES.

An increase in amplitude of the monitored electrical activity to a value greater than baseline amplitude can indicate food or drink intake. In certain embodiments, an about three to about four fold increase in amplitude from baseline amplitude indicates food or drink intake.

In some embodiments, an amplitude of about 0.30 mV to about 0.90 mV indicates a dry swallow, or an amplitude of about 0.40 mV to about 0.80 mV, about 0.45 mV to about 0.75 mV, or about 0.5 mV to about 0.7 mV indicates a dry swallow. In a particular embodiment, an amplitude of about 0.6 mV indicates a dry swallow. Alternatively, an about two-fold increase in amplitude indicates a dry swallow. A “dry swallow” is a swallow in the absence of food or drink.

In some embodiments, an amplitude of about 0.31 mV to about 1.03 mV indicates a drink intake (wet swallow), or an amplitude of about 0.43 mV to about 0.91 mV, about 0.52 mV to about 0.88 mV, or about 0.58 mV to about 0.82 mV indicates a drink intake. In a certain embodiment, an amplitude of about 0.7 mV indicates a drink intake. Alternatively, an about two-fold increase in amplitude indicates a wet swallow.

In some embodiments, an amplitude of about 0.55 mV to about 1.57 mV indicates solid food intake, or an amplitude of about 0.72 mV to about 1.4 mV, about 0.81 mV to about 1.32 mV, or about 0.89 mV to about 1.23 mV indicates solid food intake. In a particular embodiment, an about 1.06 mV indicates solid food intake. Alternatively, a greater than three-fold increase in amplitude indicates solid food intake, or an about three to about four fold increase in amplitude indicates solid food intake.

The specific amplitudes indicative of dry swallows, wet swallows and food intake will vary depending on the subject being examined. The range of amplitudes for a specific subject can be obtained by measuring the subject's background level of electrical activity while the subject is in a resting or non-swallowing state, then measuring the amplitudes when the subject is performing a dry swallow, is swallowing liquid, and is swallowing solid food. These observed amplitudes can be used to identify background electrical activity and different types of swallows when the subject is subsequently monitored for food or drink intake.

Reversion of an increased amplitude back to baseline or to a value of approximately baseline amplitude can indicate that food or drink intake has stopped. Further, a decrease in amplitude from a higher value to a lower value can indicate that food or drink intake has stopped. In some embodiments, an about three to about four fold decrease in amplitude from the increased amplitude indicates food or drink intake has stopped. In certain embodiments, an amplitude of about 0.135 mV to about 0.495 mV indicates that food or drink intake has stopped, or an amplitude of about 0.195 mV to about 0.435 mV, about 0.225 mV to about 0.405 mV, or about 0.255 my to about 0.375 mV indicates that food or drink intake has stopped. In a particular embodiment, an amplitude of about 0.315 mV indicates that food or drink intake has stopped.

Data on electrical activity in the LES can be transmitted to a recording/analyzing device, such as a microprocessor incorporated in the computerized hydraulic system, by way of electrodes. In another embodiment, a miniaturized recorder implanted in the LES or in contact with a proximate region to the LES can transmit data in a wireless fashion to an implanted system, or to an outside device.

In some embodiments, a signal indicating that a subject has started consuming food or drink, is in the process of consuming food or drink, has stopped consuming food or drink, has consumed food or drink, or any combination thereof, can be generated based on the amplitude and duration of the electrical activity of the LES or proximate to the LES. The signal can be sent to a receiving device, such as a microprocessor in the computerized hydraulic system or a system containing a receiving device, or other device or system associated with food or drink intake or the cessation of food or drink intake. As such, additional embodiments can further comprise using a receiver to receive signals regarding the subject's food or drink intake, in particular embodiments, the receiving device is used in a clinical application associated with food or drink intake. Thus, the detection of food or drink intake or cessation of food or drink intake, or signals indicative thereof, may be used in conjunction with other technology for clinical applications. That is, the detection of food or drink intake or the cessation of food or drink intake, or signals indicative thereof, can be used to trigger an intervention treatment that is associated with the food or drink intake or the cessation of food or drink intake.

Additional embodiments can further comprise using a computer or computer system to perform a number of functions, for example, including but not limited to receiving electrical signals, analyzing electrical signals, processing electrical signals, and sending a signal regarding the received, analyzed and/or processed electrical signals to another system, computer or device. Such computers and computer systems are known in the art and one of skill in the art will be able to determine, without undue experimentation, a computer or a computer system that is suitable for such use.

A device for practicing the method of detecting food or drink intake can comprise: a) one or more electrodes, for monitoring electrical activity of the subject's LIES or a region proximate to the LES; and a microprocessor that can monitor electrical activity and incorporates an algorithm that detect eating and generate electrical signals based on the monitored electrical activity. Such microprocessor can be part of a computerized hydraulic system The device can further comprise a recording module, for recording electrical data based on the monitored electrical activity. In some embodiments, one or more pairs of electrodes is utilized. The detection device can be configured to automatically detect food or drink intake in a subject.

In one embodiment, the detection device comprises one or more electrodes, and a microprocessor, wherein the one or more electrodes are connected to the microprocessor that can analyze electrical signals from the LES or a region proximate to the LES and also send electrical signals to activate the hydraulic system. The one or more electrodes can be one or more pairs of electrodes, or be a single lead (e.g., bipolar electrode). In particular embodiments, the detection device is configured to measure the amplitude and/or duration of the electrical activity in the LES or in the proximate region to the LES. In some embodiments, the detection device is an implantable device.

In one embodiment the whole system is implantable. In another embodiment, the microprocessor is positioned outside the body, and both recording of electrical signals from the LIES or a region proximate to the LES and delivery of signals to the hydraulic system can be done by wireless connections. In some embodiments, a recording of the electrical activity is obtained by placing wands on the subject's skin that detect the electrical activity, and connecting the wands to data loggers.

The electrode can be any size suitable for placement on the LES or a proximate region to the LES. In various embodiments, the electrodes can be about 1 mm long to about 50 mm long, about 5 mm long to about 25 mm long, or about 10 mm long to about 20 mm long. In one embodiment, the electrode may be about 15 mm long. The electrode can be any shape suitable for placement at the LES; for example, circular, square, rectangular, etc., and can be any dimension suitable for placement at the LES.

In some embodiments, the detection device can further comprise a computer. The computer can be used to perform a number of functions; for example, including but not limited to receiving electrical signals, analyzing electrical signals, processing electrical signals, and sending a signal regarding the received, analyzed and/or processed electrical signals to another system, computer or device.

In one embodiment, the detection device is configured to generate and send a signal to another device indicating the electrical activity of the LES. In some embodiments, the signal can be a signal that indicates that the subject has started consuming food or drink, is consuming food or drink, has stopped consuming food or drink, has consumed food or drink, or any combination thereof.

In some embodiments, the detection device is configured to generate and send a signal when an increase in amplitude from baseline amplitude is detected. In another embodiment, the device is configured to generate and send a signal that the subject has consumed food or drink when an about three to about four fold increase in amplitude from a baseline amplitude is detected.

In some embodiments, the detection device is configured to generate and send a signal that the subject has swallowed when an amplitude of about 0.30 mV to about 0.90 mV has been detected. In particular embodiments, the device is configured to generate and send a signal that the subject has swallowed when an amplitude of 0.40 mV to about 0.80 mV, about 0.45 mV to about 0.75 mV, or about 0.5 mV to about 0.7 mV has been detected. In a particular embodiment, the device is configured to generate and send a signal that the subject has swallowed when an amplitude of about 0.6 mV has been detected. Alternatively, the device is configured to generate and send a signal that the subject has swallowed when an about two-fold increase in amplitude has been detected.

In some embodiments, the detection device is configured to generate and send a signal that the subject has consumed a liquid when an amplitude of about 0.31 mV to about 1.03 mV, about 0.43 mV to about 0.91 mV, about 0.52 mV to about 0.88 mV, or about 0.58 mV to about 0.82 mV has been detected. In a particular embodiment, the device is configured to generate and send a signal that the subject has consumed a liquid when an amplitude of about 0.7 mV has been detected. Alternatively, the device is configured to generate and send a signal that the subject has consumed a liquid when an about two-fold increase in amplitude has been detected.

In some embodiments, the detection device is configured to generate and send a signal that the subject has consumed solid food when an amplitude of 0.55 mV to about 1.57 mV has been detected. In certain embodiments, the device is configured to generate and send a signal that the subject has consumed food when amplitude of about 0.72 mV to about 1.4 mV, about 0.81 mV to about 1.32 mV, or about 0.89 mV to about 1.23 mV has been detected. In a particular embodiment, the device is configured to generate and send a signal that the subject has consumed food when amplitude of about 1.06 mV has been detected. Alternatively, the device is configured to generate and send a signal that the subject has consumed food when greater than a two-fold increase in amplitude has been detected. In particular embodiments, the device is configured to generate and send a signal that the subject has consumed food when an about three to about four fold increase in amplitude is detected.

Reversion of an increased amplitude back to baseline or to a value of approximately baseline amplitude can indicate that food or drink intake has stopped. Further, a decrease in amplitude from a higher value to a lower value can indicate that food or drink intake has stopped. In some embodiments, the detection device is configured to generate and send a signal that the subject has ceased consuming food or drink when a reversion of the increased amplitude back to approximately baseline amplitude is detected. In certain embodiments, the device is configured to generate and send a signal that the subject has stopped consuming food or drink when an about three to about four fold decrease in amplitude from the increased amplitude is detected.

In some embodiments, the detection device can be configured to generate and send a signal that the subject has ceased consuming food or drink when an amplitude of about 0.135 mV to about 0.495 mV has been detected. In particular embodiments, the device may be configured to generate and send a signal that the subject has ceased consuming food or drink when amplitude of about 0.195 mV to about 0.435 mV, about 0.22.5 mV to about 0.405 mV, about 0.255 mV to about 0.375 mV has been detected. In a particular embodiment, the device is configured to generate and send a signal that the subject has ceased consuming food when an amplitude of about 0.315 mV has been detected.

These signals may be useful for a variety of clinical applications. The signals may be used in conjunction with other technology for clinical applications. That is, the signal generated when food or drink intake is detected or when the cessation of food or drink intake is detected may be used to trigger an intervention treatment that is associated with the food or drink intake or the cessation of food or drink intake.

A system for practicing the method can comprise the following. A pair of electrodes is implanted in the lower esophageal sphincter (LES) at the level of the gastro-esophageal junction. The electrodes are connected to a microprocessor. The microprocessor receives and processes signals from the electrodes regarding the subject's intake of food and drink. The microprocessor can send a signal to a pump. All of these components can be manufactured as a single device.

In one embodiment, the system comprises a device for monitoring the electrical activity of the LES and a computer for interpreting and/or recording the electrical activity of the LES. In another embodiment, the system further comprises a device for recording the electrical activity of the LES. The device for monitoring the electrical activity can comprise one or more electrodes, and a microprocessor, wherein the microprocessor, can be connected to the one or more electrodes and the device is configured to measure the electrical activity in the LES or in a proximate region to the LES. In particular embodiments, the device for monitoring the electrical activity is configured to measure the amplitude and/or duration of the electrical activity in the LES or in the proximate region to the LES. The pulse generator can be used to generate pulses or signals that are read and processed by a computer.

The electrode can be any size suitable for placement at the LES. In various embodiments, the electrode can be about 1 mm long to about 50 mm long, about 5 mm long to about 25 mm long, or about 10 mm long to about 20 mm long. In one embodiment, the electrode is about 15 mm long. The electrode can be any shape suitable for placement at the LES, such as circular, square, rectangular, etc. The electrode can also be of any dimension suitable for placement at the LES.

A computer can be used to perform a number of functions, for example, including but not limited to receiving electrical signals, analyzing electrical signals, processing electrical signals, and sending a signal regarding the electrical signals to another system, computer or device

Additional embodiments of the system further comprise a receiver for receiving signals regarding a subject's food or drink intake.

The system can comprise a device for monitoring the electrical activity and a device for sending a signal to a second system or device. In one embodiment, the second system or device is a system or device for the treatment of obesity.

The device for sending a signal to a second system or device can be configured to generate and send a signal to indicate the electrical activity of the LES. In particular embodiments, the signal is a signal indicating that the subject has started consuming food or drink, is in the process of consuming food or drink, has stopped consuming food or drink, has consumed food or drink, or any combination thereof.

In some embodiments, the device for sending a signal is configured to generate and send a signal when an increase in amplitude from baseline amplitude is detected. In certain embodiments, the device is configured to generate and send a signal that the subject has consumed food or drink when an about three to about four fold increase in amplitude from a baseline amplitude is detected.

In some embodiments, the device is configured to generate and send a signal that the subject has swallowed when an amplitude of about 0.30 mV to about 0.90 mV has been detected. In particular embodiments, the device is configured to generate and send a signal that the subject has swallowed when an amplitude of 0.40 mV to about 0.80 mV, about 0.45 mV to about 0.75 mV, or about 0.5 mV to about 0.7 mV has been detected. In a particular embodiment, the device is configured to generate and send a signal that the subject has swallowed when an amplitude of about 0.6 mV has been detected. Alternatively, the device is configured to generate and send a signal that the subject has swallowed when an about two-fold increase in amplitude has been detected.

In some embodiments, the device is configured to generate and send a signal that the subject has consumed a liquid when amplitude of about 0.31 mV to about 1.03 mV has been detected. In other embodiments, the device is configured to generate and send a signal that the subject has consumed a liquid when amplitude of 0.43 mV to about 0.91 mV, about 0.52 mV to about 0.88 mV, or about 0.58 mV to about 0.82 mV has been detected. In a particular embodiment, the device is configured to generate and send a signal that the subject has consumed a liquid when amplitude of about 0.7 mV has been detected. Alternatively, the device is configured to generate and send a signal that the subject has consumed a liquid when an about two-fold increase in amplitude has been detected.

In some embodiment, the device is configured to generate and send a signal that the subject has consumed food when an amplitude of 0.55 mV to about 1.57 mV has been detected. In other embodiments, the device is configured to generate and send a signal that the subject has consumed food when an amplitude of about 0.72 mV to about 1.4 mV, about 0.81 mV to about 1.32 mV, or about 0.89 my to about 1.23 mV has been detected. In a particular embodiment, the device is configured to generate and send a signal that the subject has consumed food when an amplitude of about 1.06 mV has been detected. Alternatively, the device is configured to generate and send a signal that the subject has consumed food when greater than a two-fold increase in amplitude has been detected. In particular embodiments, the device is configured to generate and send a signal that the subject has consumed food when an about three to about four fold increase in amplitude is detected.

Reversion of an increased amplitude back to baseline or to a value of approximately baseline amplitude can indicate that food or drink intake has stopped. Further, a decrease in amplitude from a higher value to a lower value can indicate that food or drink intake has stopped. In some embodiments, the device is configured to generate and send a signal that the subject has ceased consuming food or drink when a reversion of the increased amplitude back to approximately baseline amplitude is detected. In particular embodiments, the device is configured to generate and send a signal that the subject has stopped consuming food or drink when an about three to about four fold decrease in amplitude from the increased amplitude is detected.

In some embodiment, the device is configured to generate and send a signal that the subject has ceased consuming food or drink when an amplitude of about 0.135 mV to about 0.495 my has been detected, in certain embodiments, the device is configured to generate and send a signal that the subject has ceased consuming food or drink when an amplitude of about 0.195 mV to about 0.435 mV, about 0.225 mV to about 0.405 mV, about 0.255 mV to about 0.375 mV has been detected. In a particular embodiment, the device is configured to generate and send a signal that the subject has ceased consuming food when an amplitude of about 0.315 mV has been detected.

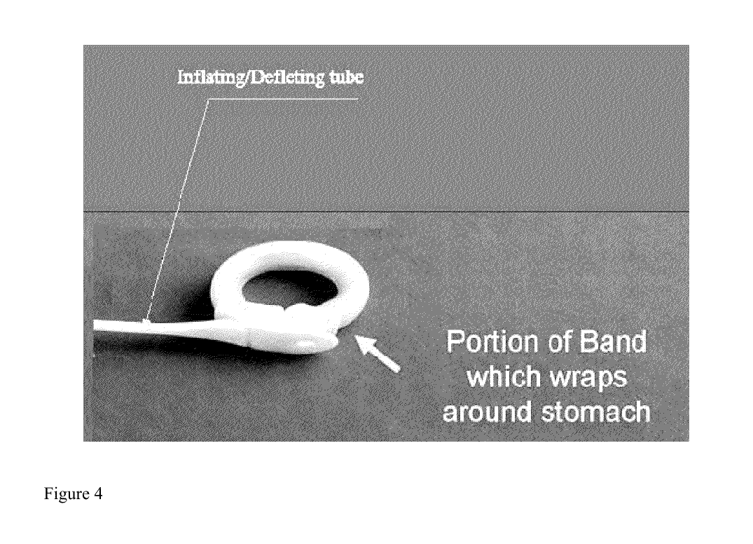

In FIG. 4, a prior art gastric band is shown. The band includes the portion that wraps around a stomach, and a tube for inflating and deflating the band.

Various embodiments are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments have been shown and described, it will be obvious to those skilled in the art that, based upon the teachings herein, changes and modifications may be made without departing, from this invention and its broader aspects. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A system for controlling a gastric band in a subject, comprising
an inflatable/deflatable gastric band;
a monitoring unit that monitors electrical activity changes of the subject's lower esophageal sphincter; and
a hydraulic system that moves fluid into and out of the gastric band in response to a signal from the monitoring unit about the electrical activity changes.

2. The system of claim 1, wherein the hydraulic system comprises a pump for moving the fluid, and one or more reservoirs for holding the fluid.

3. The system of claim 1, wherein the hydraulic system comprises a high pressure reservoir fluidly connected to the gastric band, a low pressure reservoir fluidly connected to the gastric hand, and a pump for moving fluid between the high and low pressure reservoirs.

4. The system of claim 1, wherein the monitoring unit, the hydraulic system, or both, are implantable on or within the subject.

5. The system of claim 1, wherein the monitoring unit comprises a microprocessor that monitors electrical activity and detects eating and/or generates electrical signals based on the monitored electrical activity.

6. The system of claim 1, further comprising a computer for receiving electrical signals, analyzing electrical signals, processing electrical signals, and sending a signal regarding the electrical signals to another system, computer or device.

7. The system of claim 1, further comprising one or more electrodes for detecting the electrical activity changes and positionable within, in contact with or proximate to the gastroesophageal junction of the subject.

8. The system of claim 1, wherein the electrical activity changes indicate that the subject has started consuming food or drink, is consuming food or drink, has stopped consuming food or drink, has consumed food or drink, or any combination thereof.

9. The method of claim 8, wherein an increase in amplitude of the electrical activity changes to a value greater than baseline indicates food or drink intake.

10. The method of claim 8, wherein an about three to about four fold increase in amplitude of the electrical activity changes from baseline indicates food or drink intake.

11. A method of controlling a gastric band in a subject, comprising:
moving fluid into and out of an inflatable/deflatable gastric band in response to changes in electrical activity of the subject's lower esophageal sphincter.

12. The method of claim 11, further comprising monitoring the changes in electrical activity prior to moving the fluid.

13. The method of claim 12, wherein the monitoring comprises monitoring the electrical activity by use of a microprocessor, or monitoring the electrical activity by use of one or more electrodes positioned within, in contact with or proximate to the gastroesophageal junction of the subject, or a combination thereof.

14. The method of claim 11, wherein the changes in the electrical activity indicate that the subject has started consuming food or drink, is consuming food or drink, has stopped consuming food or drink, has consumed food or drink, or any combination thereof.

15. The method of claim 14, wherein an increase in amplitude of the electrical activity changes to a value greater than baseline indicates food or drink intake.

16. The method of claim 14, wherein an about three to about four fold increase in amplitude of the electrical activity changes from baseline indicates food or drink intake.

17. The method of claim 11, wherein the subject is undergoing treatment for obesity or treatment to prevent obesity.
